(19) United States
(12) Patent Application Publication
(10) Pub. No.: US 2008/0064076 A1
(43) Pub. Date: Mar. 13, 2008

(54) Dissolved Oxygen Profile to Increase Fermentation Productivity and Economics

(76) Inventor: Victor M. Saucedo, Willowbrook, IL (US)

Correspondence Address: AIR LIQUIDE INTELLECTUAL PROPERTY DEPT., 2700 POST OAK BLVD., SUITE 1800, HOUSTON, TX, 77056, US

(21) Appl. No.: 11/746,323

(22) Filed: May 9, 2007

Related U.S. Application Data
(60) Provisional application No. 60/843,608, filed on Sep. 11, 2006.

(51) Int. Cl. C12P 7/06; C12M 1/00; C12M 1/02

(52) U.S. Cl. 435/161; 435/303.1; 435/303.3

(57) ABSTRACT

Aerobic fermentation processes and systems establish, via sensing and regulating, a particular dissolved oxygen (DO) profile throughout fermentation to improve the fermentation processes. For some embodiments, the DO profile may follow one cycle of increasing and decreasing DO level in a fermentation media from lag to stationary phases of microorganisms during the fermentation process. As one example for controlling the DO level in the media, the DO level may increase during growth phase of the microorganisms to a set maximum controlled level and then at about when a maximum growth rate of the microorganisms occurs may decrease to reach a set minimum controlled level at about when growing stops.

### CROSS-REFERENCE TO RELATED APPLICATIONS

This application claims the benefit under 35 U.S.C. §119(e) to provisional patent application No. 60/843,608, filed Sep. 11, 2006, the entire contents of which are incorporated herein by reference.

### BACKGROUND

Fermentation represents an industrial process employed to produce various fermentation products utilized by, for example, food, pharmaceutical, biotechnology, brewing and water treatment industries. Batch aerobic fermentation occurs in a reaction vessel or fermentor that contains yeast, bacteria or other aerobic microorganisms along with a carbon containing substrate for consumption by the microorganisms to produce useful products. Environmental conditions maintained in the fermentor support growth of the microorganisms.

Amount of dissolved oxygen (DO) in media within the fermentor during the aerobic fermentation affects productivity and substrate yield. While too low levels of the DO can be detrimental to the microorganisms, too high levels of the DO can inhibit growth of the microorganisms. The amount of the DO in the media depends on particular microorganism fermentations and flow rate, pressure, and concentration of oxygen in a gas supply to the fermentor. Therefore, many fermentation systems measure the DO in the media and control quantity of oxygen added to the fermentor according to a DO profile (the DO profile describes the changes in the oxygen level during fermentation). For example, some approaches maintain a constant DO level during fermentation processes. However, known DO profiles still result in excess or insufficient oxygen levels, thereby detrimentally affecting productivity and yield. In addition, these inefficient DO profiles waste the oxygen being supplied. Any wasted energy or any unused reactant added increases product unit costs and can make fermentation processes uneconomical.

Therefore, there exists a need for improved methods and apparatus for fermentation with microorganisms under aerobic fermentation conditions.

### SUMMARY

For some embodiments, a method of conducting an aerobic fermentation process includes providing a fermentation media including fermenting microorganisms and a substrate fermentable by the microorganisms and controlling a dissolved oxygen (DO) level in the fermentation media by initial increase of the DO level to a maximum target value and then, during growth of the microorganisms, beginning reduction of the DO level from the maximum target value to a minimum target value maintained thereafter through completion of the fermentation process.

In some embodiments, a method of conducting an aerobic fermentation process includes providing a fermentation media including fermenting microorganisms and a substrate fermentable by the microorganisms, and controlling a DO level in the media by increasing the DO level to reach a maximum target value at a first time, wherein the increasing occurs over a period of time beginning with a start of exponential growth of the microorganism. The method further includes controlling the DO level in the media by decreasing the DO level from the maximum target value to reach a minimum target value at a second time, wherein transition from the maximum to minimum target values begins during the growth, such as the exponential growth, of the microorganism. The method further includes substantially maintaining the DO level through completion of the fermentation process after reaching the minimum target value at the second time.

According to some embodiments, a system for conducting an aerobic fermentation process includes a fermentor and a controller configured to regulate a DO level in media within the fermentor, wherein the controller includes regulation instructions governing output signals from the controller in order to perform a method that includes increasing the DO level in the media by initial raising of the DO level to a maximum target value, and then, decreasing the DO level from the maximum target value to a minimum target value maintained thereafter through completion of the fermentation process, wherein the decreasing starts during growth of the microorganisms.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 illustrates a fermentation system that controls a dissolved oxygen (DO) level in media contained within a fermentor in order to establish a non-constant DO profile, in accordance with embodiments of the invention;

FIG. 2 illustrates a graph of exemplary fermentation process results with fermentation times identified, based on the results, for changing control of the DO level in the media, in accordance with embodiments of the invention;

FIG. 3 illustrates a plot for one example of the non-constant DO profile, according to an embodiment of the invention; and

FIG. 4 illustrates a flow chart of a method of conducting a fermentation process of aerobic microorganisms and with a non-constant DO profile, according to embodiments of the invention.

### DESCRIPTION OF PREFERRED EMBODIMENTS

Embodiments generally relate to aerobic fermentation processes such as batch fermentation processes and systems that establish, via sensing and regulating, a particular dissolved oxygen (DO) profile throughout fermentation to improve the fermentation processes. Teachings described herein extend to any industrial fermentation process, such as employed by food, pharmaceutical, biotechnology, brewing and water treatment industries. The fermentation processes occur in a reaction vessel or fermentor that contains yeast, bacteria or other aerobic microorganisms along with a carbon containing substrate for consumption by the microorganisms to produce useful products. For some embodiments, the DO profile may follow one cycle of increasing and decreasing DO level in a fermentation media from lag to stationary phases of microorganisms during the fermentation process. As one example for controlling the DO level in the media, the DO level may increase during a growth phase of the microorganisms to a maximum allowed DO, which is normally defined by an inhibition point of the microorganisms, and then, at about when a maximum growth rate of the microorganisms occurs, the DO level may decrease to reach DO limitation of the microorganisms at about when the growth of the microorganisms stops.

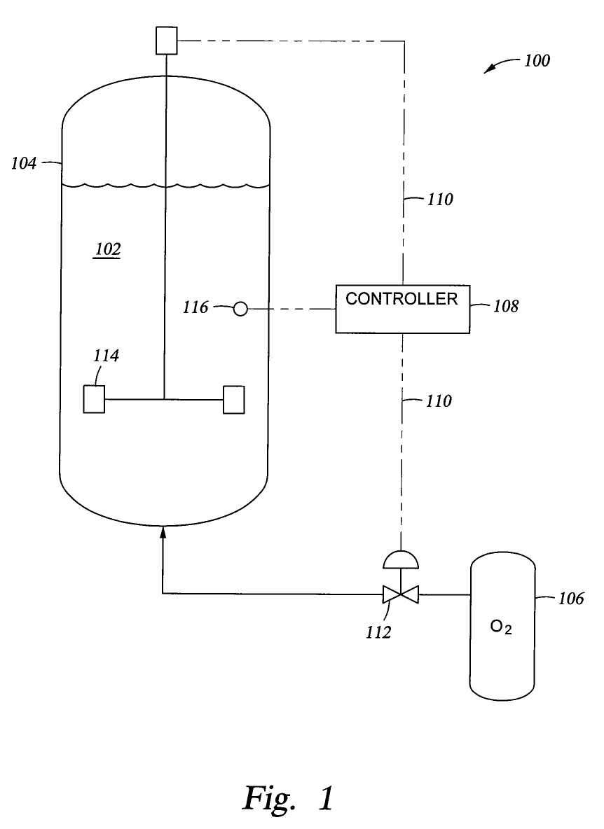

FIG. 1 shows a fermentation system 100 that controls a DO level in media 102 contained within a fermentor 104 in order to establish one or more non-constant DO profiles. The system 100 includes an O₂ supply 106 connected with the fermentor 104 and a controller 108 for regulating aspects of the system 100 to achieve a given non-constant DO profile. To this end, the controller 108 is programmed with, or programmable with, one or more non-constant DO profiles. The controller 108 may be a general-purpose computer (e.g., a workstation functioning under the control of an operating system) or a special-purpose programmable device such as a programmable logic controller (PLC). An output 110 of the controller 108 transmits control signals that actuate a valve 112 disposed between the O₂ supply 106 and the fermentor 104 to regulate a flow rate through the valve 112. The O₂ supply 106 contains oxygen which may be in the form of a gas such as air, pure oxygen or air enriched with oxygen. Increasing or decreasing the flow rate through the valve 112 respectively increases or decreases amount of oxygen supplied to the fermentor 104 in order to control the DO level as described herein.

In some embodiments, the output 110 of the controller 108 may further regulate operation of an agitator 114 disposed within the fermentor 104. For example, the agitator 114 may define a rotor that mechanically disturbs the media 102 upon the rotor being driven by a motor whose speed is governed by the control signals from the controller 108. Agitation of the media 102 enhances the DO level such that manipulating amount of agitation may also be utilized to assist in obtaining the DO profile.

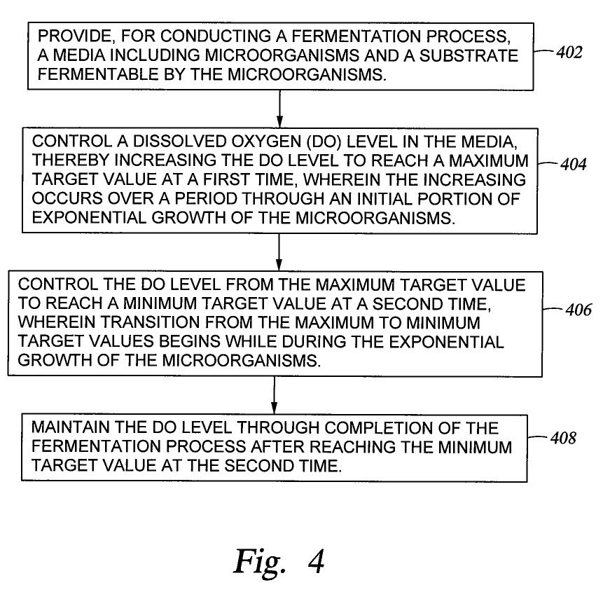

The controller 108 utilizes feedback from a DO probe 116 within the fermentor 104. The DO probe 116 measures the DO level in the media 102 to achieve proper regulation of the DO level by the controller 108. The controller 108 controls the DO level to achieve during the fermentation process the non-constant DO profile selected. The controller 108 thus may include a computer with tangible computer readable storage medium encoded with instructions to perform a method such as described herein and shown in FIG. 4. Inputting dynamics of the fermentation process into the controller 108 may further enable the controller to calculate the non-constant DO profile utilizing appropriate control algorithms based on the teachings herein.

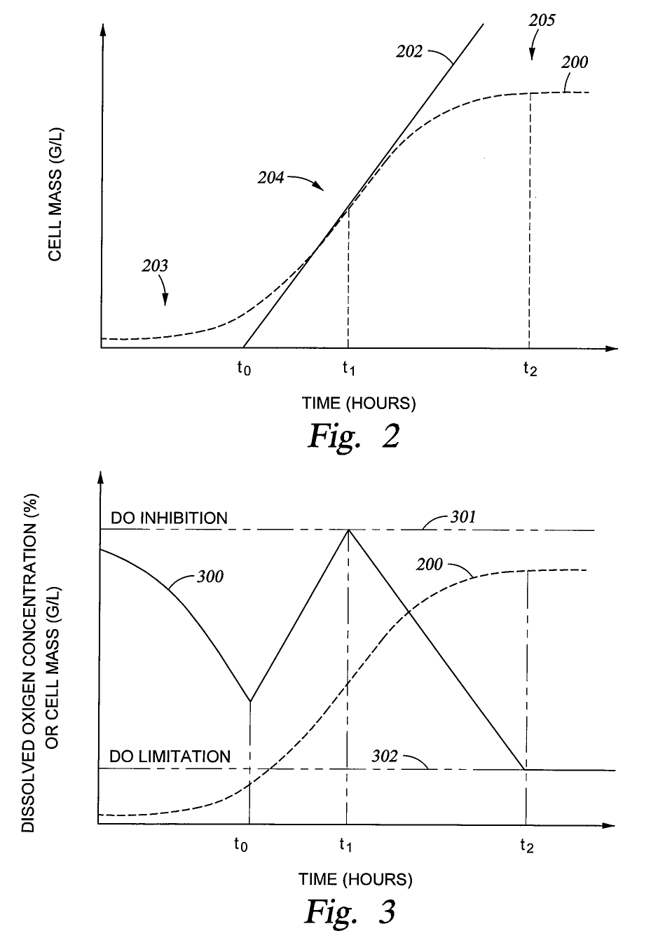

FIG. 2 illustrates a graph of exemplary fermentation process results, such as obtained by the system 100, depicted by a curve 200 plotting cell mass with respect to time. A line 202 corresponds to a highest slope along the curve 200 and hence represents a maximum growth rate during a fermentation process when microorganisms in the media 102 are growing fastest. Preliminary fermentation experiments conducted without applying the non-constant DO profile may determine cell growth rates for obtaining the curve 200. As an example, the curve 200 may be obtained with the preliminary fermentation experiments by taking samples from the media 102 at intervals of time and measuring optical density of the samples to determine cell mass since amount of light absorption is proportional to number of cells. Further, iterations from previous batch fermentations that did apply an estimated or initial non-constant DO profile may provide the preliminary fermentation experiments.

A lag phase 203, an exponential growth phase 204 and a stationary phase 205 occur throughout the fermentation process from start to end and are represented by the curve 200. Controlling the DO level in the media 102 by adjusting rate of increase or decrease of the DO level to selected values occurs at first, second and third times t₀, t₁, t₂. The curve 200 enables selection of the times t₀, t₁, t₂ that are used to define control changes along the non-constant DO profile. The first time t₀ occurs approximately when the growth phase 204 starts. For some embodiments, the first time t₀ may correspond to interception of the line 202 with the x-axis, which represents time, such that the interception identifies a specific point in time assigned to the first time t₀. The second time t₁ occurs during the growth phase 204 intermediate the lag and stationary phases 203, 205. In some embodiments, the second time t₁ corresponds to when the maximum growth rate occurs. The third time t₂ occurs at about the end of the growth phase 204, such as when the growth rate stops or the stationary phase 205 is reached. In one embodiment, the maximum growth rate (second time t₁) and cessation of the growth rate (third time t₂) may be determined according to the derivative of the curve 200.

FIG. 3 shows a plot for one example of a non-constant DO profile 300. During the lag phase 203, natural reduction of the DO level from saturated conditions occurs since no injection of the oxygen from the O₂ supply 106 replenishes the oxygen being consumed. The natural reduction of the DO level continues until the first time t₀ when injection of oxygen starts. At the first time t₀, the controller 108 actuates the valve 112 and/or operates the agitator 114 to increase over time the DO level to a maximum target value 301, such as a DO inhibition level as known or experimentally determined previously for the microorganisms. Operation of the controller 108 occurs automatically and may function to establish an increase of the DO level from the first time t₀ such that the maximum target value 301 is reached at the second time t₁. For some embodiments, the maximum target value 301 may represent an arbitrary highest value or be defined by physical conditions of the system 100, such as any safety or operational requisites like flooding.

Upon reaching the second time t₁, the controller 108 changes control criteria to begin reduction of the DO level from the maximum target value 301 to a minimum target value 302 (e.g., an arbitrary lowest value or a DO limitation as known or experimentally determined previously for the microorganisms) that is maintained through completion of the fermentation process. The controller 108 establishes rate of decrease from the second time t₁ such that the minimum target value 302 is reached at the third time t₂. The DO level maintained at the minimum target value 302 once in the stationary phase 205 avoids killing the microorganisms without wasting O₂ since the microorganisms are no longer growing.

The DO profile 300 follows one cycle of increasing and decreasing the DO level in the media 102 from lag to stationary phases 203, 205. The cycle sets the target values 301, 302 that the DO profile 300 reaches at certain times during the fermentation process. As long as the DO profile 300 reaches the maximum and minimum target values 301, 302 at respectively the second and third times t₁, t₂ and begins downward trending from the second to third times t₁, t₂ while still in the growth phase 204, the DO profile 300 may follow a direct or indirect path to the target values 301, 302. While linear upward and downward slopes are respectively depicted along the DO profile 300 between respectively the first and second times t₀, t₁ and the second and third times t₁, t₂, other shaped slopes such as second or third order curves may form the DO profile 300 between corresponding ones of the times t₀, t₁, t₂. For example, the rate of change in the DO level (as controlled by the controller 108) may be constant (as shown in FIG. 3) or may vary (e.g., the rate may initially increase and then gradually decrease as the second time is reached). Thus, the change in the level may be linear or non-linear or a combination of both for a given transition between time periods (e.g., from t₀ to t₁).

Illustratively, the DO level is shown in FIG. 3 increasing linearly at a given rate. However, it is understood that the given rate at which the DO level increases may be different for different embodiments. Further, while FIG. 3 shows a linear increase of the level (i.e., a fixed rate of increase), it is also contemplated that the rate at which the level increases may vary between the various time periods (e.g., between the first time t₀ and the second time t₁). For example, the rate may initially increase at the first time t₀, and then the rate may gradually decrease as the time approaches the second time t₁.

The DO profile 300 improves positive effects of oxygen transfer and utilization when most needed by setting the maximum for the DO level to coincide with fastest growth of the microorganisms. Thereafter, decreasing the DO level additionally improves positive effects of oxygen transfer and utilization by avoiding wasting unused oxygen in the media 102 at the end of the fermentation process. During part of the growth phase 204 leading into the stationary phase 205, rate of oxygen addition decreases, instead of continuously increasing, without causing detriment to the growth of the microorganisms. The DO profile 300 depicts controlling the DO level independent of quantity of cells to avoid oxidant, such as oxygen, waste since concentration of cells fails to provide indication of growth that requires sufficient levels of oxygen to not inhibit productivity. Relative to constant maintaining of the DO level through the fermentation process, the non-constant DO profile 300 contributes to productivity of the system 100 by not limiting total oxygen available to the microorganisms when needed yet not wasting oxygen either due to increased likelihood that oxygen is not even transferred to the media 102 from input of the O₂ supply 106 or resulting from left over oxygen at the end of the fermentation process.

FIG. 4 illustrates a flow chart of a method of conducting a fermentation process with a non-constant DO profile. The fermentation process begins at an initial step 402 by providing a media that includes aerobic microorganisms and a carbon containing substrate for consumption by the microorganisms during fermentation. A DO ramping up step 404 involves manipulating oxidant input and/or agitation of the media to increase a DO level in the media in accordance with selected profile criteria, such as set forth herein. Increasing the DO level occurs at least over a period of time through an initial portion of exponential growth of the microorganisms in order to reach a maximum target value at a first time. Next, further manipulation occurs at DO reduction step 406 to decrease the DO level from the maximum target value to reach a minimum target value at a second time. The decrease begins while during the growth, such as the exponential growth, of the microorganisms. At end step 408, maintaining the DO level after reaching the minimum target value at the second time continues through to the completion of the fermentation process.

Operation of the controller 108 based on the foregoing criteria improves both yield and productivity of the system 100. Manipulating flow from the O₂ supply 106 to the fermentor 104 with the controller 108 to achieve, for example, the DO profile 300 improves amount of product or cells produced for a given quantity of substrate in the media 102. In addition to improving how many cells are produced relative to how much of the substrate is consumed, the productivity achieved with the system 100 results in improved number of cells or product grown per unit time.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method of conducting an aerobic fermentation process, comprising:
a) providing a fermentation media including fermenting microorganisms and a substrate fermentable by the microorganisms; and
b) controlling a dissolved oxygen (DO) level in the fermentation media by initial increase of the DO level to a predetermined maximum target value and then, during growth of the microorganisms, beginning reduction of the DO level from the maximum target value to a predetermined minimum target value maintained thereafter through completion of the fermentation process.

2. The method of claim 1, wherein the minimum target value is about at a lower DO limitation for the microorganisms.

3. The method of claim 1, wherein the maximum target value is about at an upper DO inhibition for the microorganisms.

4. The method of claim 1, wherein the minimum target value is about at a lower DO limitation for the microorganisms and the maximum target value is about at an upper DO inhibition for the microorganisms.

5. The method of claim 1, wherein the increase and reduction occur with a substantially linear rate.

6. The method of claim 1, wherein the initial increase begins at a time about when an exponential growth phase of the microorganisms starts.

7. The method of claim 1, wherein the beginning reduction occurs at about a time associated with a maximum growth rate of the microorganisms.

8. The method of claim 1, wherein the initial increase begins at a first time about when an exponential growth phase of the microorganisms starts and the beginning reduction occurs at about a second time associated with a maximum growth rate of the microorganisms.

9. The method of claim 1, wherein controlling the DO level includes regulating supply of oxidant to the media.

10. The method of claim 1, wherein controlling the DO level includes regulating agitation of the media.

11. The method of claim 1, further comprising monitoring the DO level in the media and adjusting the DO level to achieve the controlling based on feedback from the monitoring.

12. A method of conducting an aerobic fermentation process, comprising:
a) providing a fermentation media including fermenting microorganisms and a substrate fermentable by the microorganisms;
b) controlling a dissolved oxygen (DO) level in the media by increasing the DO level to reach a predetermined maximum target value at a first time, wherein the increasing occurs over a period of time beginning with start of exponential growth of the microorganism;
c) controlling the DO level in the media by decreasing the DO level from the maximum target value to reach a predetermined minimum target value at a second time, wherein transition from the maximum to minimum target values begins during the growth of the microorganism; and
d) maintaining the DO level through completion of the fermentation process after reaching the minimum target value at the second time.

13. The method of claim 12, wherein the transition from the maximum to minimum target values begins at the first time.

14. The method of claim 13, wherein the DO level lacks active regulation prior to the controlling the DO level in the media by increasing the DO level.

15. The method of claim 12, wherein the minimum and maximum target values are respectively a lower DO limitation for the microorganisms and an upper DO inhibition for the microorganisms.

16. The method of claim 12, wherein the increasing and decreasing occur with a linear rate.

17. A system for conducting an aerobic fermentation process, comprising:
a) a fermentor; and
b) a controller configured to regulate a dissolved oxygen (DO) level in media within the fermentor, wherein the controller includes regulation instructions governing output signals from the controller in order to perform a method, comprising:
i) increasing the DO level in the media by initial raising of the DO level to a predetermined maximum target value; and
ii) then, decreasing the DO level from the maximum target value to a predetermined minimum target value maintained thereafter through completion of the fermentation process, wherein the decreasing starts during growth of the microorganisms.

18. The system of claim 17, further comprising an oxygen source, wherein the controller is configured to regulate flow from the oxygen source to the fermentor.

19. The system of claim 17, further comprising an agitator disposed in the fermentor, wherein the controller is configured to regulate operation of the agitator.

20. The system of claim 17, further comprising a DO sensing probe within the fermentor, wherein the probe provides feedback signals to the controller.
